(19) United States
(12) Patent Application Publication
(10) Pub. No.: US 2011/0105633 A1
(43) Pub. Date: May 5, 2011

(54) FUNCTIONALIZED STILBENE-POLYALKYLENE OXIDE PREPOLYMERS

(75) Inventor: Michael T. Milbocker, Holliston, MA (US)

(73) Assignee: PROMETHEAN SURGICAL DEVICES LLC, East Hartford, CT (US)

(21) Appl. No.: 12/912,117

(22) Filed: Oct. 26, 2010

Related U.S. Application Data
(60) Provisional application No. 61/254,892, filed on Oct. 26, 2009.

(51) Int. Cl. C08G 18/00; A61F 2/44

(52) U.S. Cl. 521/82; 528/75; 523/113

(57) ABSTRACT

Stilbene-polyalkylene oxide prepolymers which include a polyalkylene oxide backbone with two or more isocyanate substituents and at least one hydroxystilbene or methoxystilbene substituent are useful as one component adhesives and biocompatible device coatings. Stilbene-polyalkylene compositions useful as a two component adhesive contain: a) a polyalkylene oxide having two or more amine substituents with b) a stilbene polyisocyanate compound, or alternatively contain: a) a polyethylene oxide having two or more isocyanate substituents with b) a stilbene diamine compound, or, alternatively contain: a) a bioabsorbable group in addition to a stilbene group attached to a polyalkylene backbone and b) a diamine compound.

### RELATED APPLICATIONS

This application claims the benefit of priority under 35 U.S.C. §119(e) of U.S. Prov. App. No. 61/254,892, filed Oct. 26, 2009, the disclosure of which is incorporated herein by reference.

### FIELD OF THE INVENTION

This disclosure relates to compounds comprising multiple polyalkylene oxide-NCO functionalized groups bonded to stilbene, such as resveratrol analogs. Such compounds can be useful as surgical adhesives, coatings, and in situ curing tissue modifying structures.

### BACKGROUND

A number of polyalkylene glycol-based polymer systems such as prepolymers of polyurethane and polyureaurethane are known. For example, poloxamers (e.g., Pluronics®) are known in the art as block copolymers containing ethylene oxide and propylene oxide monomers. Most polymerizing compositions comprising polyalkylene-oxides are hydrophobic and require a two-part composition containing excessive NCO that do not incorporate water into the polymerized product. Successful in situ polymerizing compositions are hydrophilic to enhance biocompatibility, one part to enhance use, contain a minimal percentage NCO to reduce toxicity, and incorporate water directly into the polymerized mass in volumetric ratios as high as 99% and not less than 25%.

There remains a need for new materials having at least one of the following properties: capable of metabolically-acceptable in situ polymerization, biodegradable, resistant to oxidative degradation in the body, and having therapeutic benefits.

### SUMMARY OF THE INVENTION

Disclosed herein is a compound comprising two or more isocyanate-capped polyalkylene glycols, the polyalkylene glycols being bonded to one or both aryl groups of a stilbene via a urea linkage. In one embodiment, the compound comprises 2-4 isocyanate-capped polyalkylene glycols, e.g., 3 isocyanate-capped polyalkylene glycols.

In one embodiment, the compound is formed from a reaction between a stilbene reactant having two or more hydroxyl groups on one or both aryl groups, and a polyalkylene glycol diisocyanate. In one embodiment, the stilbene reactant is selected from: 3,5-dihydroxystilbene, 3,3′,4,5′-tetrahydroxystilbene, 3,4,4′,5-tetrahydroxystilbene, 3,3′,5,5′-tetrahydroxystilbene, 3,3′,4,5,5′-pentahydroxystilbene, 3,5-dimethoxystilbene, 3,4′,5-trimethoxystilbene, 3,3′,4,5′-tetramethoxystilbene, 3,4,4′,5-tetramethoxystilbene, 3,3′,5′5′-tetramethoxystilbene, and 3,3′,4,5,5′-pentamethoxystilbene.

Another embodiment provides a compound comprising two or more amine-capped polyalkylene glycols, the polyalkylene glycols being bonded to one or both aryl groups of a stilbene via a urea linkage.

In one embodiment, the polyalkylene glycols can be a combination of ethylene oxide and propylene oxide to form a random or block copolymer as the backbone. Exemplary backbones have molecular weights ranging from 500 to 20,000, e.g., from 1000 to 10,000, or from 1000 to 5000. In one embodiment, the two or more polyalkylene glycols are triols comprising from 60 to 80% ethylene oxide and from 20 to 40% propylene oxide, by number. In one embodiment, the two or more polyalkylene glycols are terminated with two toluene diisocyanate groups, and the stilbene is 3,5,4′-trihydroxystilbene, wherein the resulting structure presents 3 functional NCO groups. In another embodiment, the two or more polyalkylene glycols are terminated with three toluene diisocyanate groups and the stilbene is 3,5,4′-trihydroxystilbene and the resulting structure contains 6 functional NCO groups. In yet another embodiment, the two or more polyalkylene glycols are terminated with three isophorone diisocyanate groups, and the stilbene is 3,5,4′-trihydroxystilbene and the resulting structure contains 6 functional NCO groups.

In one embodiment, the compound further comprises at least one biodegradable group substituted between an isocyanate group of the urea linkage and at least one of the two or more polyalkylene glycols. In one embodiment, the at least one biodegradable group is derived from a compound selected from glycolic acid, glycolide, lactic acid, lactide, ε-caprolactone, p-dioxanone, trimethylene carbonate, and substituted alkylene carbonates.

Other embodiments provide a composition comprising the compound described herein (e.g., two or more isocyanate-capped polyalkylene glycols, the polyalkylene glycols being bonded to one or both aryl groups of a stilbene via a urea linkage). In one embodiment, the composition can comprise a) the isocyanate-capped polyalkylene oxide-stilbene compound, as described herein and b) amine-terminated polyalkylene glycols.

In another embodiment, the composition comprises a) the amine-capped polyalkylene oxide-stilbene compound, as described herein, and b) a compound comprising an isocyanate-capped polyalkylene glycol containing at least two isocyanate groups.

In any of these embodiments, the composition can further comprise free molecular diisocyanate. Also disclosed is a foam comprising the compositions described herein mixed with equal parts by volume of an aqueous solution, or a gel comprising the compositions described herein mixed with 1 to 5% by volume aqueous solution.

### DETAILED DESCRIPTION

Useful in situ polymerizing compositions can be constructed by endcapping linear or multifunctional (e.g., trifunctional) polyalkylene oxide chains with diisocyanate. In the case of the linear chains, the durability of the polymerized composition can be enhanced by reacting three NCO endcapped linear chains with a triol comprised of three OH groups. In one embodiment, a resveratrol analog is substituted for the triol. These substitutions provides one or more benefits, including: (1) the potential for providing a biodegradable linkage; (2) the potential for enhancing oxidation resistance; and (3) the possibility of providing a therapeutic benefit to an organism implanted with the substituted polymer.

In one embodiment, an in situ polymerizing compound is provided herein, based on polyalkylene oxide-stilbene components comprising a polyalkylene oxide (polyalkylene glycol) backbone with at least one isocyanate substituent and at least one urea linkage to a stilbene. In one embodiment, the compound comprises two or more isocyanate-capped polyalkylene glycols, where the polyalkylene glycols are bonded to one or both aryl groups of a stilbene via a urea linkage.

In one embodiment, the compound has the following formula: [NCO-I-urethane-D-urethane]ₙ-S where “I” is an isocyanate center (e.g. toluene in a toluene diisocyanate), “D” is a diol center comprised of polyalkylene oxide (polyalkylene glycol) blocks, and n is the number of substituents bonded to a stilbene “S” via its aryl rings. The total “n” substituents can be bonded to one or both stilbene aryl rings. For example, the source of “S” can be a stilbenehydroxyl compound (e.g., a resveratrol analog) having n hydroxyl (OH) or methoxyl groups prior to polymerization to stilbene center S, wherein all the OH groups formerly existing on the diols and stilbene are transformed into urethane links.

Exemplary isocyanate centers for “I” include those derived from diisocyanates such as toluene diisocyanate, phenylene diisocyanate, hexamethylene diisocyanate, isophorone diisocyanate, xylylene diisocyanate, cyclohexylene diisocyanate, and methylene diphenyl diisocyanate. Diol center D, can comprise polyalkylene oxide blocks as described in greater detail herein.

In one embodiment, this compound is a resveratrol analog which can be useful as a biocompatible coating and tissue adhesive where the polyalkylene oxide backbone is attached to the each OH group of resveratrol via urea linkages. In the case of resveratrol the resulting structure contains at least three active NCO groups. When the polyalkylene backbone is linear the resulting compound with resveratrol is 3-functional. When the polyalkylene backbone is trifunctional with 3 NCO groups the functionality of the resulting compound with resveratrol is 6-functional.

Any resveratrol analog can be used, e.g., stilbene having two or more hydroxylated or methoxylated groups, such as 2-10, 2-6, 2-5, or 2-4 groups, e.g., 3 groups (trifunctional). Examples include 3,5-dihydroxystilbene, 3,3′,4,5′-tetrahydroxystilbene, 3,4,4′,5-tetrahydroxystilbene, 3,3′,5,5′-tetrahydroxystilbene, 3,3′,4,5,5′-pentahydroxystilbene, 3,5-dimethoxystilbene, 3,4′,5-trimethoxystilbene, 3,3′,4,5′-tetramethoxystilbene, 3,4,4′,5-tetramethoxystilbene, 3,3′,5′5′-tetramethoxystilbene, and 3,3′,4,5,5′-pentamethoxystilbene. These form prepolymers with NCO terminated polyalkylene chains with NCO functionality equal to the number of OH groups on the resveratrol analog. When the resveratrol analog contains 3 or more OH groups it is not necessary that every OH group be attached to a polyalkylene chain, although the open OH groups may associate with the free NCO groups over time. Compositions that consume all the OH groups have enhanced shelf-life.

In another embodiment wherein the polyalkylene oxide backbone has a branched or multi-arm structure, at least one arm from each branched polyalkylene backbone is attached to an OH group on a polyalkylene oxide-stilbene compound, e.g., a resveratrol analog.

In another embodiment an absorbable composition useful as a two component adhesive is provided by combining a) a polyalkylene oxide having two or more amine substituents with b) the polyalkylene oxide stilbene compound described herein, e.g., a resveratrol analog diisocyanate compound. In one embodiment, the polyalkylene oxide having two or more amine substituents includes a polyalkylene oxide backbone that can be branched or multi-armed with an amine substituent at the end of each arm. For example, a resveratrol analog diisocyanate compound is formed by attaching a diisocyanate to each OH group of the resveratrol analog.

In yet another embodiment an absorbable composition useful as a two component adhesive is provided by combining a) a polyalkylene oxide having two or more isocyanate substituents with b) a resveratrol analog diamine terminated compound. In one embodiment, the polyalkylene oxide having two or more isocyanate substituents includes a polyalkylene oxide backbone that can be branched or multi-armed. For example, a resveratrol analog diamine terminated compound can include the addition of polyalkylene arms to reduce viscosity as described above with respect to the previous two component adhesive embodiments. The rate of bioabsorbability could be adjusted by the addition of groups derived from any monomer known to form a bioabsorbable polymer (including but not limited to glycolic acid, glycolide, lactic acid, lactide, 1,4-dioxane-2-one, 1,3-dioxane-2-one, ε-caprolactone and the like) or groups derived from a diacid which will provide an absorbable linkage (including but not limited to succinic acid, adipic acid, malonic acid, glutaric acid, sebacic acid, diglycolic acid and the like).

The polyalkylene oxide-stilbene compound (e.g., resveratrol-based) in situ polymerizing compounds and compositions described herein are useful as surgical adhesives and/or tissue augments for joining, replacing or enhancing portions of body tissue.

The polyalkylene oxide-stilbene compounds (e.g., resveratrol-based) described herein are useful as surgical adhesives and tissue augments and include a polyalkylene oxide backbone substituted with one or more isocyanate groups. The polyalkylene oxide backbone can be derived from any alkylene oxide although the overall hydrophobicity will affect its compatibility with water. In one embodiment, the backbone is copolymeric possessing a block structure, arranged randomly or ordered in the backbone, at least one block type being hydrophilic and another block type being hydrophilic. The ratio and distribution of such blocks can be controlled to achieve a desired compatibility with water, which can be a factor in designing compounds that form hydrogels and bond to wet tissue during in situ polymerization.

An example of a useful polyalkylene oxide backbone can include ethylene oxide, polyethylene oxide (PEO) backbone, and mixtures and blocks thereof. As another example, the polyalkylene oxide backbone can be a propylene oxide to form a polypropylene oxide (PPO) backbone. In one embodiment, a combination of ethylene oxide and propylene oxide can be used to form a random or block copolymer as the backbone. The molecular weight of the polyalkylene oxide backbone should be chosen to provide desired physical characteristics to the final compound. Exemplary backbones have molecular weights ranging from 500 to 20,000, e.g., from 1000 to 10,000, or from 1000 to 5000.

In certain embodiments, the polyalkylene oxide backbone has a branched or multi-arm structure. For example, the polyalkylene oxide backbone can be the result of polymerizing alkylene oxide monomer in the presence of a multi-functional (e.g., polyhydric) initiator. Reaction conditions for producing branched or multi-arm polyalkylene oxide backbones are known to those skilled in the art.

Other embodiments provide compositions comprising the compounds described herein. In one embodiment, the composition further comprises free molecular diisocyanate, e.g., diisocyanates selected from toluene diisocyanate, phenylene diisocyanate, hexamethylene diisocyanate, isophorone diisocyanate, xylylene diisocyanate, cyclohexylene diisocyanate, and methylene diphenyl diisocyanate. The free molecular diisocyanate can be present in an amount ranging from 0.1 to 5% by weight of the composition, from 0.1 to 3% by weight of the composition, or from 0.1 to 1.5% by weight of the composition.

In one embodiment the resveratrol-based compound is synthesized by reacting linear chains comprised of 75% polyethylene oxide and 25% polypropylene oxide by number having a molecular weight of approximately 1000 Dalton with a diisocyanate. For example, the diisocyanate is aromatic such as toluene diisocyanate. The result is NCO terminated polyalkylene chains with mean molecular weight less than 2000 Dalton. These chains are trimerized by the addition of resveratrol (3,5,4′-trihydroxystilbene). The result is trifunctional molecules comprised of three polyalkylene arms terminated with NCO and centered on resveratrol.

In another embodiment, the resveratrol-based compound is synthesized by reacting trifunctional molecules comprised of 75% polyethylene oxide and 25% propylene oxide by number having a molecular weight of approximately 4500 Dalton with diisocyanate. For example, the diisocyanate is aromatic such as toluene diisocyanate. The result is NCO terminated trifunctional polyalkylene oxide with mean molecular weight less than 9000 Dalton. These structures are further trimerized by the addition of 3,5,4′-trihydroxystilbene. The result is 6-functional molecules comprised of three trifunctional polyalkylene molecules terminated with NCO and centered on resveratrol.

The resveratrol of the above two compositions can be substituted with any of the following resveratrol analogs: 3,3′,4,5′-tetrahydroxystilbene, 3,4,4′,5-tetrahydroxystilbene, 3,3′,5,5′-tetrahydroxystilbene, 3,3′,4,5,5′-pentahydroxystilbene, 3,5-dimethoxystilbene, 3,4′,5-trimethoxystilbene, 3,3′,4,5′-tetramethoxystilbene, 3,4,4′,5-tetramethoxystilbene, 3,3′,5′5′-tetramethoxystilbene, and 3,3′,4,5,5′-pentamethoxystilbene. The resulting geometry of these compositions depends on the number of available OH groups on the substituted resveratrol analog. In one embodiment, the ratio of reactants are chosen such that all OH groups are consumed. This provides a prepolymer with enhanced shelf-life.

Mixtures of linear and trifunctional polyalkylene oxides are also contemplated. The ratio in this instance may be selected to achieve a desired prepolymer viscosity. The viscosity of higher functional prepolymers tend to have a higher viscosity. Similarly, the ratio of reactants may be chosen such that a slight excess of molecular diisocyanate remains after synthesis which tends to lower prepolymer viscosity, ensure complete consumption of OH groups, and enhance tissue bonding.

In one embodiment, the number of isocyanate groups present on the polyalkylene oxide backbone is selected to provide desired physical characteristics to the compound upon exposure to moisture. A greater degree of substitution will provide greater cross-linking which will provide a material that exhibits less swelling and less compliance. A lower degree of substitution will yield a less cross-linked material having greater compliance.

The present compounds can be prepared by reacting a polyalkylene oxide backbone having two or more hydroxyl groups with a molar excess of diisocyanate to provide a polyalkylene diisocyanate. On to this structure resveratrol analog groups can be added by alternating addition of resveratrol and diisocyanate. In this case a resveratrol analog containing two hydroxyl groups is desired. For example, 3,5-dimethoxystilbene. In this way an bioabsorbable group is added to the polyalkylene oxide backbone. Other absorbable groups may also be added as are known in the art. They may be directly bonded to the polyalkylene oxide to attached via diisocyanate linkages. While the exact reaction conditions will depend upon the specific starting components, generally speaking the polyalkylene oxide backbone and the diisocyanate are reacted at temperatures in the range of 40° C. to 80° C., for a period of time from 24 hours to 72 hours at atmospheric pressure.

The present polyalkylene oxide-stilbene compounds (e.g., resveratrol-based compounds) can be used as single component adhesives or tissue augments. These compositions may be mixed with water prior to implantation or applied directly to tissue. Polymerization occurs the composition is exposed to water. Alternatively, a tertiary amine may be used to initiate polymerization. The present one-part compositions polymerize when water reacts with the isocyanate groups to form a corresponding amine and carbon dioxide. The amine reacts with additional isocyanate located on the polyalkylene or with excess molecular diisocyanate present in the composition to form polyurea which foams due to the simultaneous evolution of carbon dioxide thereby forming a porous, polymeric bridge. The degree of porosity can be increased by increasing the % NCO of the prepolymer composition. This can be done by decreasing the molecular weight of the prepolymer while keeping the functionality constant.

The compositions disclosed herein cross-link in conditions normally found in the body. The rate of cross-linking will vary depending on a number of factors such as the particular diisocyanate compound employed, the degree of isocyanate substitution, the hydrophilicity of the alkylene oxide backbone and the functionality of the prepolymer. Normally, the cross-linking reaction is conducted at temperatures ranging from 20° C. to about 50° C. for thirty seconds to about one hour or more. The amount of water employed will normally range from about 50% by volume to 99% by volume. While water is a typical reactant to effect cross-linking it should be understood that other compounds could also be employed either together with or instead of water. Such compounds include diethylene glycol, polyethylene glycol and diamines, such as, for example, diethylamino propanediol.

When the polyalkylene oxide-stilbene compound (e.g., resveratrol-based) is intended for implantation it is possible to effectuate cross-linking in situ using the water naturally present in a mammalian body or with supplemented water. However, to more precisely control the conditions or to reduce the time the prepolymer remains a liquid in the body, it may be advantageous to partially cross-link the compound prior to its use as an implant. This is most easily accomplished by premixing the prepolymer with water. This approach can also be used to remove some of the liberated carbon dioxide from the composition before implantation.

The polyalkylene oxide-stilbene compound (e.g., resveratrol-based) described herein can also be cross-linked by the application of water vapor or diamine vapor. These compositions readily dissolve in organic solvents, and can be used to reduce the viscosity of a prepolymer by the addition of acetone or toluene. These cross-linking and dilutive techniques can be useful when the compounds are to be used in coating surgical mesh, e.g., when the coating method involves dipping of the substrate in prepolymer.

In another embodiment a composition useful as a tissue adhesive or coating is two-part. Accordingly, disclosed herein are compositions comprising the polyalkylene oxide-stilbene compounds disclosed herein in addition to a polyalkylene oxide having one or more amine substituents.

The amine-terminated polyalkylene oxide can be derived from any alkylene oxide and can be homopolymeric or copolymeric. For example, the amine-substituted polyalkylene oxide can be derived from ethylene oxide and be an amine-substituted polyethylene oxide (PEO). As another example, the polyalkylene oxide can be derived from propylene oxide and be an amine-substituted polypropylene oxide (PPO). As yet another example, a combination of ethylene oxide and propylene oxide can be used to form a random or block copolymer as the amine-substituted polyalkylene oxide. In one embodiment, the number ratio of ethylene oxide to propylene oxide ranges from 50 to 80% ethylene oxide. The molecular weight of the amine-substituted polyalkylene oxide can be chosen to provide desired physical characteristics to the final composition. The lower the molecular weight the higher the prepolymer viscosity. In one embodiment, the backbones have molecular weights ranging from 500 to 20,000, e.g., from 1000 to 10,000, or from 1000 to 5000.

In certain embodiments, the polyalkylene oxide backbone has a branched or multi-arm structure. For example, the polyalkylene oxide backbone can be the result of polymerizing alkylene oxide monomer in the presence of a multi-functional (e.g., polyhydric) initiator. Reaction conditions for producing branched or multi-arm polyalkylene oxide backbones are known to those skilled in the art.

The amine groups are terminally located on the polyalkylene oxide arms. More specifically, amine groups are to be substituted wherever a hydroxyl group resides on the polyalkylene backbone. Likewise, although a single amine group per polyalkylene oxide arm can be used, it is also contemplated that more than one amine group per polyalkylene oxide arm may be present. The substituted amine groups may be multi-functional.

The number of amine groups present on the polyalkylene oxide backbone is selected to provide desired physical characteristics to the compound upon exposure to a second component containing isocyanate groups. A greater degree of substitution will provide greater cross-linking which will provide a material that exhibits less swelling and less compliance. A lower degree of substitution will yield a less cross-linked material having greater compliance. The degree of cross-linking is also dependent upon the molar ratio of amine groups to isocyanate groups. In order to consume all amine groups in the polymerization it is desirable for the mixture to contain an excess number of isocyanate groups. In addition to forming cross-links within the composition, isocyanate groups can also bond to amine groups in living tissue. In one embodiment, when these compositions are used as tissue adhesives, it is desirable to have at least 1% excess isocyanate. The excess is arrived at by calculating the % NCO=A required to consume all amine groups in the mixture and adjusting the composition so that the prepolymerized % NCO=A+1. The % NCO can be adjusted by increasing the relative proportion of isocyanate substituted polyalkylene oxide to amine substituted polyalkylene oxide, one or more also containing a resveratrol analog substitution. Alternatively, molecular diisocyanate may be added to the mixture. This alternative adjustment will also aid in reducing the viscosity of the mixture.

The preparation of amine-substituted polyalkylene oxides is within the purview of those skilled in the art.

The amine-substituted polyalkylene oxide is optionally combined with one or more alkylene oxide-stilbene compounds, e.g., resveratrol analogs. This is accomplished by attaching isocyanate groups to the hydroxyl groups of the stilbene compound followed by reacting the resulting polyisocyanate with an amine-substituted polyalkylene oxide. The molar ratio of isocyanate groups to amine groups should be chosen to leave some amine groups unaffected. For example, a desired molecular weight can be achieved by introducing an amount of polyisocyanate such that a desired final molecular structure is achieved. These calculations are known in the art, and are based on assuming all the NCO groups of the polyisocyanate react with amine groups of the amine-substituted polyalkylene oxide.

In certain embodiments, the polyalkylene oxide-stilbene compounds disclosed herein (e.g., the resveratrol polyisocyanate) that is combined with the amine-substituted polyalkylene oxide includes a polyalkylene oxide backbone substituted with one or more isocyanate groups.

Another embodiment provides a compound comprising two or more amine-capped polyalkylene glycols, the polyalkylene glycols being bonded to one or both aryl groups of a stilbene via a urea linkage. In another embodiment, disclosed herein are compositions comprising the amine-capped polyalkylene oxide-stilbene compounds in addition to a compound comprising an isocyanate-capped polyalkylene glycol containing at least two isocyanate groups.

The polyalkylene oxide backbone can be derived from any alkylene oxide and can be homopolymeric or copolymeric. Thus, for example, the polyalkylene oxide backbone can be derived from ethylene oxide and be a polyethylene oxide (PEO) backbone. As another example, the polyalkylene oxide backbone can be derived from propylene oxide and be a polypropylene oxide (PPO) backbone. As yet another example, a combination of ethylene oxide and propylene oxide can be used to form a random or block copolymer as the backbone. The molecular weight of the polyalkylene oxide backbone should be chosen to provide desired physical characteristics to the final compound. Exemplary backbones have molecular weights ranging from 500 to 20,000, e.g., from 1000 to 10,000, or from 1000 to 5000.

In certain embodiments, the polyalkylene oxide backbone has a branched or multi-arm structure. For example, the polyalkylene oxide backbone can be the result of polymerizing alkylene oxide monomer in the presence of a multi-functional (e.g., polyhydric) initiator. Reaction conditions for producing branched or multi-arm polyalkylene oxide backbones are known to those skilled in the art.

In all formulations using amine-substituted or isocyanate-capped polyalkylene oxide, the bioabsorbability of the polymerized product can be enhanced by the addition of known biodegradable (bioabsorbable) groups. The bioabsorbable group can be derived from a compound selected from the group consisting of glycolic acid, glycolide, lactic acid, lactide, ε-caprolactone, p-dioxanone, and trimethylene carbonate or substituted alkylene carbonates such as dimethyl trimethylene carbonate.

Those skilled in the art will readily envision reaction schemes for preparing useful bioabsorbable isocyanates. For example, bioabsorbable diisocyanates can be prepared by first preparing a bioabsorbable oligomer and then endcapping with isocyanate. Methods for the production of bioabsorbable oligomers and isocyanate endcapping are within the purview of those skilled in the art.

It will be understood that various modifications may be made to the embodiments disclosed herein. For example, the compositions in accordance with this disclosure can be blended with other polyalkylene oxide stilbene compounds, bioabsorbable groups or non-bioabsorbable materials. Therefore, the above description should not be construed as limiting, but merely as exemplifications. Those skilled in art will envision other modifications within the scope and spirit of the claims appended hereto.

### Example 1

Eight hundred and eighty grams (880 g) of polyether polyol, UCON 75-H-450 (Dow Chemical, New York), is mixed with 273 g toluene diisocyanate (Sigma Aldrich, Saint Louis, Mo.) and brought to 70° C. while mixing until the % NCO is 5.2%. Then 15.6 g resveratrol (CS Inc, Danbury , Conn.) is added while mixing, and heated until NCO 4.82%. The result is a pre-polymer useful in the replacement of disc nucleus subsequent to a disectomy, or any other application where a tissue in the body requires augmentation.

### Example 2

Seven hundred grams (8700 g) of polyether polyol, Multranol 9199 (Bayer Chemical, New York), is mixed with 85 g toluene diisocyanate (Sigma Aldrich, Saint Louis, Mo.) and brought to 70° C. while mixing until the % NCO is 3.1%. Then 11.8 g resveratrol (CS Inc, Danbury, Conn.) is added while mixing, and heated until NCO 2.5%. The result is a pre-polymer useful in the replacement of disc nucleus subsequent to a disectomy, or any other application where a tissue in the body requires augmentation.

1. A compound comprising two or more isocyanate-capped polyalkylene glycols, the polyalkylene glycols being bonded to one or both aryl groups of a stilbene via a urea linkage.

2. The compound of claim 1, wherein the compound comprises 2-4 isocyanate-capped polyalkylene glycols.

3. The compound of claim 1, wherein the compound comprises 3 isocyanate-capped polyalkylene glycols.

4. The compound of claim 1, wherein the compound is formed from a reaction between a stilbene reactant having two or more hydroxyl groups on one or both aryl groups, and a polyalkylene glycol diisocyanate.

5. The compound of claim 1, wherein the stilbene reactant is selected from:
3,5-dihydroxystilbene, 3,3′,4,5′-tetrahydroxystilbene, 3,4,4′,5-tetrahydroxystilbene, 3,3′,5,5′-tetrahydroxystilbene, 3,3′,4,5,5′-pentahydroxystilbene, 3,5-dimethoxystilbene, 3,4′,5-trimethoxystilbene, 3,3′,4,5′-tetramethoxystilbene, 3,4,4′,5-tetramethoxystilbene, 3,3′,5′5′-tetramethoxystilbene, and 3,3′,4,5,5′-pentamethoxystilbene.

6. The compound of claim 1, wherein the two or more polyalkylene glycols are triols comprising from 60 to 80% ethylene oxide and from 20 to 40% propylene oxide, by number.

7. The compound of claim 6, wherein the two or more polyalkylene glycols are terminated with two toluene diisocyanate groups, and the stilbene is 3,5,4′-trihydroxystilbene, wherein the resulting structure presents 3 functional NCO groups.

8. The compound of claim 6, wherein the two or more polyalkylene glycols are terminated with three toluene diisocyanate groups and the stilbene is 3,5,4′-trihydroxystilbene and the resulting structure contains 6 functional NCO groups.

9. The compound of claim 6, wherein the two or more polyalkylene glycols are terminated with three isophorone diisocyanate groups, and the stilbene is 3,5,4′-trihydroxystilbene and the resulting structure contains 6 functional NCO groups.

10. The compound of claim 1, further comprising at least one biodegradable group substituted between an isocyanate group of the urea linkage and at least one of the two or more polyalkylene glycols.

11. The compound of claim 10, wherein the at least one biodegradable group is derived from a compound selected from glycolic acid, glycolide, lactic acid, lactide, c-caprolactone, p-dioxanone, trimethylene carbonate, and substituted alkylene carbonates.

12. A composition comprising the compound of 1, wherein the composition further comprises free molecular diisocyanate.

13. The composition of claim 12, wherein the free molecular diisocyanate is selected from toluene diisocyanate, phenylene diisocyanate, hexamethylene diisocyanate, isophorone diisocyanate, xylylene diisocyanate, cyclohexylene diisocyanate, and methylene diphenyl diisocyanate.

14. A composition comprising a) the compound of claim 1, and b) a compound comprising amine-terminated polyalkylene glycols.

15. The composition of claim 14, further comprising free molecular diisocyanate.

16. A foam comprising the composition of claim 12 mixed with equal parts by volume of an aqueous solution.

17. A gel comprising the composition of claim 12 mixed with 1 to 5% by volume aqueous solution.

18. A compound comprising two or more amine-capped polyalkylene glycols, the polyalkylene glycols being bonded to one or both aryl groups of a stilbene via a urea linkage.

19. A composition comprising a) the compound of claim 18, and b) a compound comprising an isocyanate-capped polyalkylene glycol containing at least two isocyanate groups.

20. The composition of claim 19, further comprising free molecular diisocyanate.
